(19) United States
(12) Patent Application Publication
(10) Pub. No.: US 2016/0157990 A1
(43) Pub. Date: Jun. 9, 2016

(54) ENDOVASCULAR FENESTRATED STENT-GRAFTING

(71) Applicant: ENDOSPAN LTD., Herzilyia Pituach (IL)

(72) Inventors: Alon SHALEV, Ra'anana (IL); Sagi RAZ, Tel Aviv (IL)

(73) Assignee: ENDOSPAN LTD., Herzilyia Pituach (IL)

(21) Appl. No.: 15/019,512

(22) Filed: Feb. 9, 2016

Related U.S. Application Data
(62) Division of application No. 13/513,397, filed on Sep. 14, 2012, now abandoned.
(60) Provisional application No. 61/265,793, filed on Dec. 2, 2009.

(51) Int. Cl. A61F 2/07; A61F 2/954

(52) U.S. Cl. CPC A61F 2/07; A61F 2/954; A61F 2002/061

(57) ABSTRACT

A branching stent-graft is deployed such that a distal end thereof is in a branching blood vessel, a proximal end thereof extends into a main blood vessel, and a coupling portion thereof transitions to a partially-radially-expanded state, in which the coupling portion defines a sharp tip at a proximal end of the branching stent-graft. A main stent-graft is transvascularly introduced into the main blood vessel in a radially-compressed state. After the branching stent-graft is deployed, and while the sharp tip remains stationary, the main stent-graft is transitioned to a radially-expanded state, such that the sharp tip punctures the main covering element as the main stent-graft transitions to the radially-expanded state, thereby forming a fenestration in the main covering element. The coupling portion is transitioned to a more-radially-expanded state, so as to form a blood-impervious seal between a branching covering element and a main covering element.

### CROSS-REFERENCE TO RELATED APPLICATIONS

The present patent application is a divisional of U.S. application Ser. No. 13/513,397, filed Sep. 14, 2012, which is the U.S. national stage of International Application PCT/IL2010/001018, filed Dec. 2, 2010, which claims priority from U.S. Provisional Application 61/265,793, filed Dec. 2, 2009, entitled, “System for endovascular fenestrated stent-grafting and method for using same,” which is incorporated herein by reference.

### FIELD OF THE APPLICATION

This present application relates generally to prostheses and surgical methods, and specifically to tubular prostheses, including endovascular grafts and stent-grafts, and surgical techniques for using the prostheses to maintain patency of body passages such as blood vessels, and treating aneurysms.

### BACKGROUND OF THE APPLICATION

Endovascular prostheses are sometimes used to treat aortic aneurysms. Such treatment includes implanting a stent or stent-graft within the diseased vessel to bypass the anomaly. An aneurysm is a sac formed by the dilation of the wall of the artery. Aneurysms may be congenital, but are usually caused by disease or, occasionally, by trauma. Aortic aneurysms which commonly form between the renal arteries and the iliac arteries are referred to as abdominal aortic aneurysms (“AAAs”). Other aneurysms occur in the aorta, such as thoracic aortic aneurysms (“TAAs”) and aortic uni-iliac (“AUI”) aneurysms.

PCT Publication WO 2008/107885 to Shalev et al., and US Patent Application Publication 2010/0063575 to Shalev et al. in the US national stage thereof, which are incorporated herein by reference, describe a multiple-component expandable endoluminal system for treating a lesion at a bifurcation, including a self expandable tubular root member having a side-looking engagement aperture, and a self expandable tubular trunk member comprising a substantially blood impervious polymeric liner secured therealong. Both have a radially-compressed state adapted for percutaneous intraluminal delivery and a radially-expanded state adapted for endoluminal support.

The following references may be of interest:

U.S. Pat. No. 4,938,740

U.S. Pat. No. 5,824,040 to Cox et al.

U.S. Pat. No. 7,044,962 to Elliott

US Patent Application Publication 2006/0229709 to Morris et al.

US Patent Application Publication 2006/0241740 to Vardi et al,

US Patent Application Publication 2008/0109066 to Quinn

Fonseca A et al., “Intravascular ultrasound assessment of the novel AngioSculpt scoring balloon catheter for the treatment of complex coronary lesions,” J Invasive Cardiol 20(1):21-7 (January 2008)

### SUMMARY OF APPLICATIONS

Some applications of the present invention provide a multi-component stent-graft system. The stent-graft system comprises a main stent-graft, which is configured to be positioned in a main blood vessel, and one or more branching stent-grafts, which are configured to be positioned partially in respective branching blood vessels that branch from the main blood vessel. For example, the main blood vessel may be an aorta, and the branching blood vessels may be the renal arteries. For some applications, the stent-graft system is used for treating an abdominal aortic aneurysm.

Each of the branching stent-grafts typically comprises a support structure, which is shaped so as to define at least a coupling portion. The coupling portion is configured to transition to a partially-radially-expanded state upon deployment of the stent-graft from a tubular delivery shaft. In the partially-radially-expanded state, the coupling portion defines a sharp tip at the proximal end of the support structure.

The branching stent-grafts are positioned in the branching blood vessels such that their sharp tips extend into the main blood vessel. The main stent-graft, while in a radially-compressed state in a delivery shaft, is advanced into the main blood vessel in a vicinity of the branching blood vessels. Upon being released from the delivery shaft, the main stent-graft transitions to a radially-expanded state. As the main stent-graft radially expands, the sharp tips of the branching stent-grafts puncture a covering element of the main stent-graft, thereby forming respective fenestrations in the covering element.

One or more coupling-end expansion tools are provided for transitioning the coupling portions of the branching stent-grafts from their partially-radially-expanded states to more-radially-expanded states. The coupling-end expansion tool(s) are advanced into the branching stent-grafts, and are used to expand the coupling portions of the branching stent-grafts. As the coupling portions transition from their partially-radially-expanded states to their more-radially-expanded state, the coupling portions typically enlarge the respective fenestrations previously made in the covering element of the main stent-graft by the sharp tips of the coupling portions. Blood-impervious seals are formed between covering elements of the branching stent-grafts and the covering element of the main stent-graft.

Because the branching stent-grafts are separately deployed, each can be readily positioned in one of the branching blood vessels (e.g., renal arteries which generally branch from the main blood vessel (e.g., the aorta) at different respective axial positions along the main blood vessel. In contrast, if the main stent-graft itself were to comprise branching tubular structures, it would often be difficult to insert these tubular structures into the branching blood vessels (e.g., renal arteries). In addition, it could be necessary to use a plurality of guidewires, which would increase the crossing profile of the deployment tool.

For some applications, the support structure of each of the branching stent-grafts comprises a plurality of structural stent elements, which include a plurality of proximal structural stent elements. The proximal structural stent elements have respective proximal ends and are disposed around a longitudinal axis of the coupling portion of the branching stent-graft. The proximal ends of the proximal structural stent elements together define the sharp tip when the coupling portion is in the partially-radially-expanded state.

For some applications, the covering element of each of the branching stent-grafts is shaped so define at least one non-covered portion of the coupling portion. This non-covered portion of the coupling portion may serve to allow access into the branching stent-graft for the coupling-end expansion tool.

Typically, the coupling portion of each of the stent-grafts is configured to be initially restrained in the partially-radially-expanded state upon the deployment of the branching stent-graft from the delivery shaft. For some applications, a coupling-end restraining element is provided, which is configured to initially restrain the proximal ends of the proximal structural stent elements that together define the sharp tip.

For some applications, each of the branching stent-grafts further comprises the coupling-end restraining element, which may be fixed to a portion of the coupling portion. For example, the coupling-end restraining element may comprise at least one curved element, such as a ring, a helix, a coil, a spiral, or a corkscrew, which is fixed to at least one of the proximal structural stent elements, typically in a vicinity of the proximal end of the at least one of the proximal structural stent elements. The other ones of the proximal structural stent elements are initially threaded through the curved element, such that the coupling-end restraining element initially restrains the coupling portion in the partially-radially-expanded state after the deployment of the branching stent-graft from the delivery shaft.

For some applications, the coupling-end expansion tool comprises a radially-expandable member, and, typically, a guidewire, over which the radially-expandable member is advanced. For example, the radially-expandable member may comprise an inflatable element, such as a balloon, or a radially-expandable metal wireframe, which may, for example, comprise a shape memory alloy, e.g., Nitinol.

Although the multi-component stent-graft system is generally described herein as being applicable for placement in the area of the bifurcations of the renal arteries from the abdominal aorta, for some applications the stent-graft system is instead placed in another area of a main body lumen and one or more branching body lumens, such as a main blood vessel and one or more branching blood vessels.

There is therefore provided, in accordance with an application of the present invention, apparatus for use with a tubular delivery shaft, the apparatus including:

a stent-graft, which is configured to initially be placed in the delivery shaft in a radially-compressed state, and which includes:
a support structure, which has proximal and distal ends, and which is shaped so as to define at least a coupling portion, which is configured to transition to a partially-radially-expanded state upon deployment of the stent-graft from the delivery shaft, in which state the coupling portion defines a sharp tip at the proximal end of the support structure; and
a covering element, which is securely attached and covers at least a portion of the support structure; and

a coupling-end expansion tool, which is configured to transition the coupling portion from the partially-radially-expanded state to a more-radially-expanded state.

For some applications, when the coupling portion is in the partially-radially-expanded state, the proximal end of the support structure is disposed within a circle perpendicular to a longitudinal axis of the stent-graft, which circle has a diameter of no more than 1 mm.

For some applications, the coupling portion has (a) a partially-radially-expanded volume of at least 30 mm3, when in the partially-radially-expanded state, and (b) a more-radially-expanded volume equal to at least 300% of the partially-radially-expanded volume, when unconstrained in the more-radially-expanded state.

For some applications, a perimeter of the coupling portion monotonically decreases from a distal end thereof to a proximal end thereof when the coupling portion is unconstrained in the partially-radially-expanded state.

For some applications, the covering element covers a covered portion of the coupling portion, which covered portion extends from a distal end of the coupling portion along between 20% and 100%, such as between 20% and 75% (e.g., between 20% and 50%), of an axial distance between the distal end and a proximal end of the coupling portion, when the coupling portion is in the partially-radially-expanded state.

For some applications, the covering element is shaped so define at least one non-covered portion of a surface defined by the coupling portion, which non-covered portion has a surface area of at least 1.5 mm2, when the coupling portion is unconstrained in the partially-radially-expanded state.

For some applications, the sharp tip is oriented in a direction that is parallel to a central longitudinal axis of the stent-graft. For some applications, the sharp tip is disposed within 1 mm of the central longitudinal axis.

For some applications, the coupling portion is outwardly flared in a proximal direction when the coupling portion is unconstrained in the more-radially-expanded state.

For some applications, the support structure includes a plurality of structural stent elements, which include a plurality of proximal structural stent elements, which have respective proximal ends and are disposed around a longitudinal axis of the coupling portion, which proximal ends together define the sharp tip when the coupling portion is in the partially-radially-expanded state. For some applications, the support structure is configured such that the proximal ends of the proximal structural stent elements together do not define the sharp tip when the coupling portion is in the more-radially-expanded state. For some applications, when the coupling portion is in the partially-radially-expanded state, the proximal end of the support structure is disposed within a circle perpendicular to a longitudinal axis of the stent-graft, which circle has a diameter of no more than 1 mm. For some applications, the coupling portion is configured to be initially restrained in the partially-radially-expanded state upon the deployment of the stent-graft from the delivery shaft.

For some applications, the stent-graft further includes a coupling-end restraining element, which is configured to initially restrain the proximal ends of the proximal structural stent elements that together define the sharp tip.

For some applications, the coupling-end restraining element includes at least one curved element selected from the group consisting of: a ring, a helix, a coil, a spiral, and a corkscrew, which curved element is fixed to at least one of the proximal structural stent elements within 2 mm of the proximal end of the at least one of the proximal structural stent elements; and the other ones of the proximal structural stent elements are initially threaded through the curved element, such that the coupling-end restraining element initially restrains the coupling portion in the partially-radially-expanded state after the deployment of the stent-graft from the delivery shaft.

For some applications, the proximal structural stent elements are initially bundled together, so as to initially restrain the coupling portion in the partially-radially-expanded state after the deployment of the stent-graft from the delivery shaft.

For some applications, the coupling portion is configured to be initially restrained in the partially-radially-expanded state upon the deployment of the stent-graft from the delivery shaft. For some applications, the apparatus further includes a coupling-end restraining element, which is configured to initially restrain the coupling portion in the partially-radially-expanded state after the deployment of the stent-graft from the delivery shaft. For some applications, the stent-graft includes the coupling-end restraining element, which is coupled to at least a portion of the coupling portion. For some applications, the coupling-end restraining element includes a biologically-compatible glue. For some applications, the coupling-end restraining element is initially removably coupled to a portion of the coupling portion, and the coupling-end expansion tool is configured to decouple the coupling-end restraining element from the portion of the coupling portion. For some applications, the support structure includes a plurality of interconnected metal structural stent elements, the coupling-end restraining element includes one or more of the structural stent elements, and the coupling-end expansion tool is configured to transition the coupling portion by breaking the structural stent elements of the coupling-end restraining element.

For some applications, the coupling-end expansion tool includes a radially-expandable member, which may include, for example, an inflatable element, and/or a radially-expandable metal wireframe.

For some applications, the coupling-end expansion tool includes a guidewire. For some applications, the coupling-end expansion tool includes a guidewire and a radially-expandable member, which is configured to slide over the guidewire.

For some applications, the support structure is shaped so as to further define a distal body portion, a proximal end of which is joined to a distal end of the coupling portion, and which distal body portion is configured to transition to a radially-expanded state upon deployment of the stent-graft from the delivery shaft. For some applications, a distal end of the distal body portion coincides with the distal end of the support structure. For some applications, the distal body portion has a greatest perimeter of between 10 and 50 mm when the distal body portion is unconstrained in the radially-expanded state For some applications, the distal body portion has an axial length of between 0.5 and 5 cm when the distal body portion is in the radially-expanded state. For some applications, the stent-graft has an axial length of between 0.5 and 6 cm when the distal body portion is in the radially-expanded state and the coupling portion is in the more-radially-expanded state For some applications, the distal body portion is generally tubular when unconstrained in the radially-expanded state. For some applications, the covering element covers at least 75% of an axial length of the distal body portion, when the distal body portion is in the radially-expanded state.

For some applications, the coupling portion has a greatest perimeter of between 10 and 50 mm when the coupling portion is unconstrained in the more-radially-expanded state. For some applications, the coupling portion has an axial length of between 0.5 and 4 cm when the coupling portion is in the more-radially-expanded state. For some applications, the coupling portion has an axial length of between 0.3 and 4 cm when the coupling portion is in the partially-radially-expanded state.

For some applications, the support structure includes a metal. For example, the metal may be selected from the group consisting of: a super-elastic metal, and a shape memory alloy. For some applications, the metal includes Nitinol.

For some applications, the support structure is self-expanding.

For some applications, the apparatus further includes an extracorporeal unit, configured to transmit energy to the coupling portion, which is configured to convert the energy to thermal energy to increase a capability of the sharp tip. For some applications, the extracorporeal unit is configured to wirelessly transmit the energy.

For any of the applications described above, the stent-graft may be a branching stent-graft, and the apparatus may further include a main endovascular stent-graft, which includes a main support structure and a main covering element attached to the main support structure so as to at least partially cover the main support structure; the sharp tip may be configured to puncture the main covering element, thereby forming a fenestration in the main covering element; and the coupling portion, upon transitioning from the partially-radially-expanded state to the more-radially-expanded state, may be configured to form a blood-impervious seal between the covering element of the branching stent-graft and the main covering element.

For some applications, the coupling portion is configured to enlarge the fenestration as the coupling portion transitions from the partially-radially-expanded state to the more-radially-expanded state.

For some applications, the main stent-graft is configured to assume radially-compressed and radially-expanded states, and, when unconstrained in the radially-expanded state, has a greatest perimeter that is at least 150% of a greatest perimeter of the branching stent-graft when the coupling portion is unconstrained in the more-radially-expanded state.

For some applications, the branching stent-graft is a first branching stent-graft, and the fenestration is a first fenestration, and further including a second branching stent-graft, having the features of the first branching stent-graft described above; the sharp tip of the second branching stent-graft is configured to puncture the main covering element, thereby forming a second fenestration in the main covering element; and the coupling portion of the second branching stent-graft, upon transitioning from the partially-radially-expanded state to the more-radially-expanded state, is configured to form a blood-impervious seal between the covering element of the second branching stent-graft and the main covering element.

For some applications, the main stent-graft is configured to be placed in a main artery, and the branching stent-graft is configured to placed partially in a branch of the main artery, such that the sharp tip is within the main artery.

For any of the applications described above, the apparatus may further include the delivery shaft.

There is further provided, in accordance with an application of the present invention, a method for treating a patient, the method including:

transvascularly introducing a tubular delivery shaft partially into a branching blood vessel that branches from a main blood vessel, while a branching stent is positioned in the tubular delivery shaft in a radially-compressed state, which branching stent-graft includes a branching support structure and a branching covering element that at least partially covers a coupling portion of the branching support structure;

deploying the branching stent from the delivery shaft such that a distal end of the branching stent-graft is positioned in the branching blood vessel, and a proximal end of the branching stent-graft extends into the main blood vessel, and (b) the coupling portion transitions to a partially-radially-expanded state, in which the coupling portion defines a sharp tip at the proximal end of the branching support structure;

transvascularly introducing a main endovascular stent-graft into the main blood vessel in a radially-compressed state, which main stent-graft includes a main support structure and a main covering element attached to the main support structure so as to at least partially cover the main support structure;

transitioning the main stent-graft to a radially-expanded state, such that the sharp tip punctures the main covering element, thereby forming a fenestration in the main covering element; and

after transitioning the main stent-graft, transitioning the coupling portion of the branching stent-graft from the partially-radially-expanded state to a more-radially-expanded state, so as to form a blood-impervious seal between the branching covering element and the main covering element.

For some applications, the main and branching blood vessels are an aorta and a renal artery, respectively; transvascularly introducing the tubular delivery shaft includes transvascularly introducing the tubular delivery shaft partially into the renal artery; and transvascularly introducing the main endovascular stent-graft includes transvascularly introducing the main endovascular stent-graft into the aorta in the radially-compressed state.

For some applications, the method further includes identifying that the patient suffers from an aneurysm, and transvascularly introducing the main stent-graft includes transvascularly introducing the main stent-graft responsively to the identifying. For some applications, the method further includes identifying that the patient suffers from an aneurysm, and transvascularly introducing the branching stent-graft includes transvascularly introducing the branching stent-graft responsively to the identifying.

For some applications, transitioning the coupling portion includes enlarging the fenestration by transitioning the coupling portion.

For some applications, when the coupling portion is in the partially-radially-expanded state, the proximal end of the branching support structure is disposed within a circle perpendicular to a longitudinal axis of the branching stent-graft, which circle has a diameter of no more than 1 mm.

For some applications, the covering element is shaped so define at least one non-covered portion of a surface of the coupling portion, which non-covered portion has a surface area of at least 1.5 mm2, when the coupling portion is unconstrained in the partially-radially-expanded state.

For sonic applications, the branching support structure includes a plurality of structural stent elements, which include a plurality of proximal structural stent elements, which have respective proximal ends and are disposed around a longitudinal axis of the coupling portion, and the proximal ends of the proximal structural stent elements together define the sharp tip when the coupling portion is in the partially-radially-expanded state. For some applications, transitioning the coupling portion includes transitioning the coupling portion to the more-radially-expanded state such that the proximal ends of the proximal structural stem elements together do not define the sharp tip.

For some applications, the coupling portion is configured to be initially restrained in the partially-radially-expanded state upon the deployment of the branching stent-graft from the delivery shaft.

For some applications, the branching stent-graft further includes a coupling-end restraining element, and deploying the branching stent-graft includes deploying the branching stent-graft such that the coupling-end restraining element initially restrains the proximal ends of the proximal structural stent elements that together define the sharp tip. For some applications, the coupling-end restraining element includes at least one curved element selected from the group consisting of: a ring, a helix, a coil, a spiral, and a corkscrew, which curved element is fixed to at least one of the proximal structural stent elements within 2 mm of the proximal end of the at least one of the proximal structural stent elements; and the other ones of the proximal structural stent elements are initially threaded through the curved element, such that the coupling-end restraining element initially restrains the coupling portion in the partially-radially-expanded state after the deployment of the branching stent-graft from the delivery shaft.

For some applications, the proximal structural stent elements are initially bundled together, so as to initially restrain the coupling portion in the partially-radially-expanded state after the deployment of the branching stent-graft from the delivery shaft.

For some applications, transitioning the coupling portion includes using a coupling-end expansion tool to transition the coupling portion from the partially-radially-expanded state to the more-radially-expanded state. For some applications, the covering element is shaped so define at least one non-covered portion of the coupling portion when the coupling portion is unconstrained in the partially-radially-expanded state, and using the coupling-end expansion tool includes passing the coupling-end expansion tool through the non-covered portion.

For some applications, the branching support structure is shaped so as to further define a distal body portion, a proximal end of which is joined to a distal end of the coupling portion, and deploying the branching stent includes deploying the branching stent such that the distal body portion transitions to a radially-expanded state.

For some applications, the branching support structure is self-expanding.

For some applications, the method further includes transmitting energy from an extracorporeal unit to the coupling portion, which is configured to convert the energy to thermal energy to increase a capability of the sharp tip.

For any of the applications described above, the coupling portion may be configured to be initially restrained in the partially-radially-expanded state upon the deployment of the branching stent-graft from the delivery shaft

For any of the applications described above:

the branching stent-graft may be a first branching stent-graft, the coupling portion may be a first coupling portion, the branching blood vessel may be a first branching blood vessel, the branching support structure may be a first branching support structure, the sharp tip may be a first sharp tip, the fenestration may be a first fenestration, and the seal may be a first seal,

the method may further include, before transitioning the main stent-graft to the radially-expanded state, transvascularly introducing and deploying a second branching stent-graft such that (a) a distal end of the second branching stent-graft is positioned in a second branching blood vessel that branches from the main blood vessel, and a proximal end of the second branching stent-graft extends into the main blood vessel, and (b) a second coupling portion of the second branching stent-graft transitions to a partially-radially-expanded state, in which the second coupling portion defines a second sharp tip at a proximal end of a second branching support structure of the second branching stent-graft,

transitioning the main stent-graft may include transitioning the main stent-graft to the radially-expanded state, such that the first and second sharp tips puncture the main covering element, thereby forming the first fenestration and a second fenestration, respectively, in the main covering element, and

the method may further include, after transitioning the main stent-graft, transitioning the second coupling portion of the second branching stent-graft from the partially-radially-expanded state to a more-radially-expanded state, so as to form a second blood-impervious seal between the second branching covering element and the main covering element.

The present invention will be more fully understood from the following detailed description of embodiments thereof, taken together with the drawings, in which:

### BRIEF DESCRIPTION OF THE DRAWINGS

FIGS. 1 and 2 are schematic illustrations of a multi-component stent-graft system in disassembled and assembled states, respectively, in accordance with an application of the present invention;

FIGS. 3A-C are schematic illustrations of a branching stent-graft of the stent-graft system of FIGS. 1 and 2, in different levels of radial compression, in accordance with an application of the present invention;

FIG. 4 is a schematic illustration of a support structure of the branching stent-graft of FIGS. 3A-C unconstrained in a more-radially-expanded state, in accordance with an application of the present invention;

FIGS. 5A-D are schematic illustrations of a coupling-end expansion tool, in accordance with respective applications of the present invention;

FIGS. 6A-D are schematic illustrations of a procedure for expanding a coupling portion of a branching stent-graft of the stent-graft system of FIGS. 1 and 2, in accordance with an application of the present invention; and

FIGS. 7A-M are schematic illustrations of an exemplary transluminal delivery procedure for implanting the multi-component stent-graft system of FIGS. 1 and 2, in accordance with an application of the present invention.

### DETAILED DESCRIPTION OF APPLICATIONS

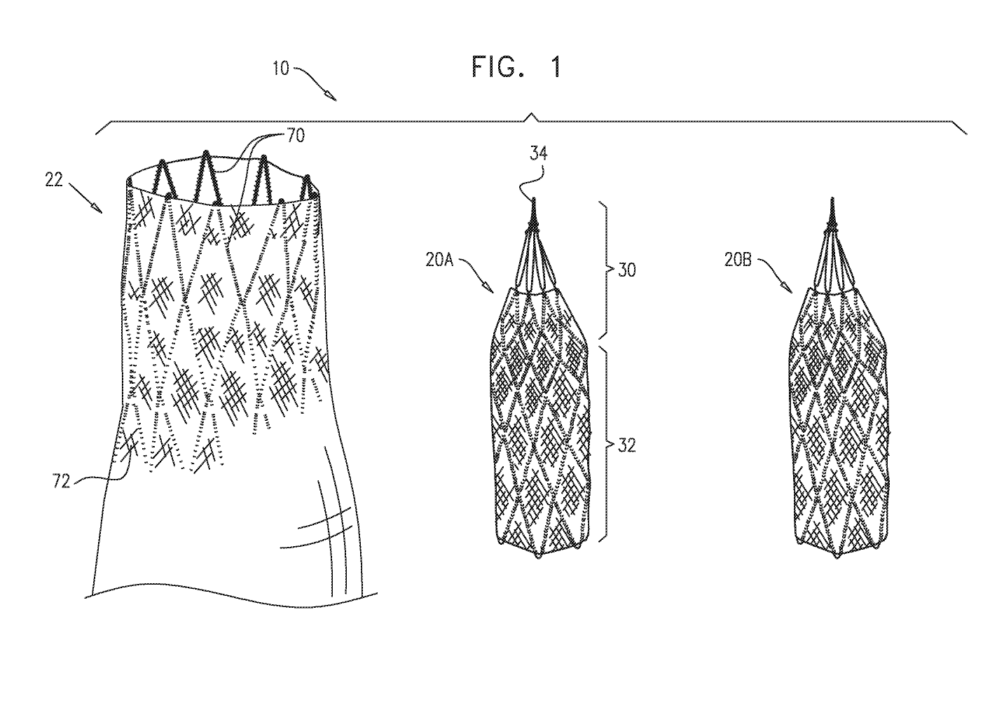

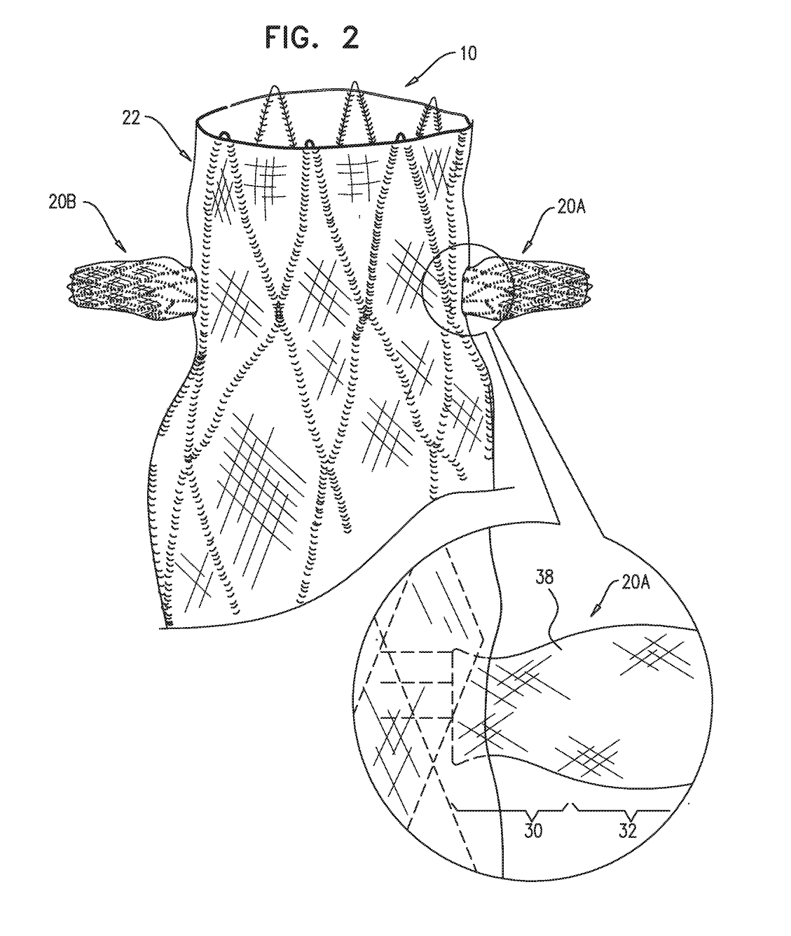

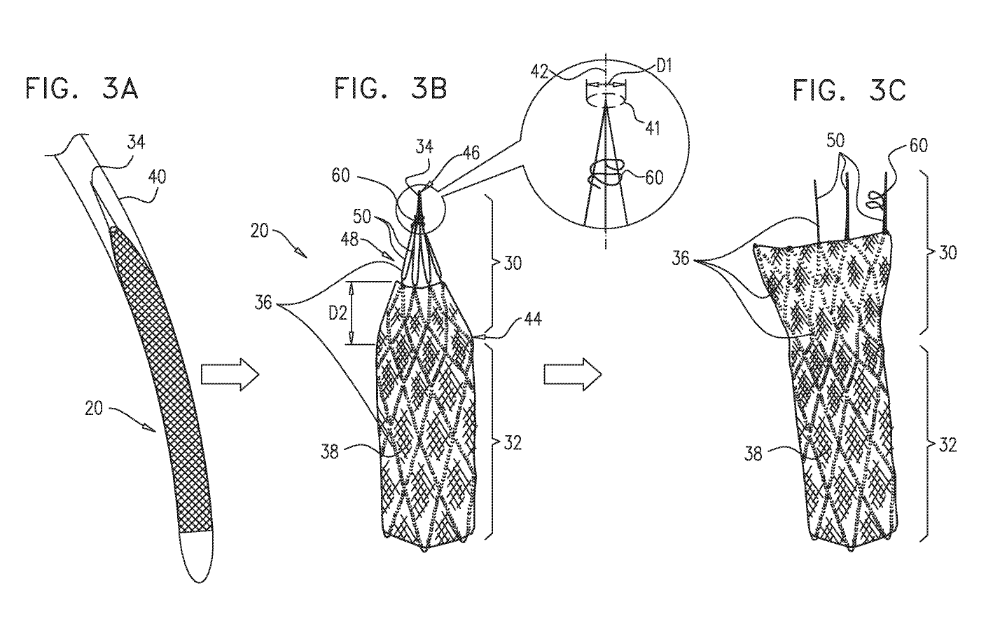

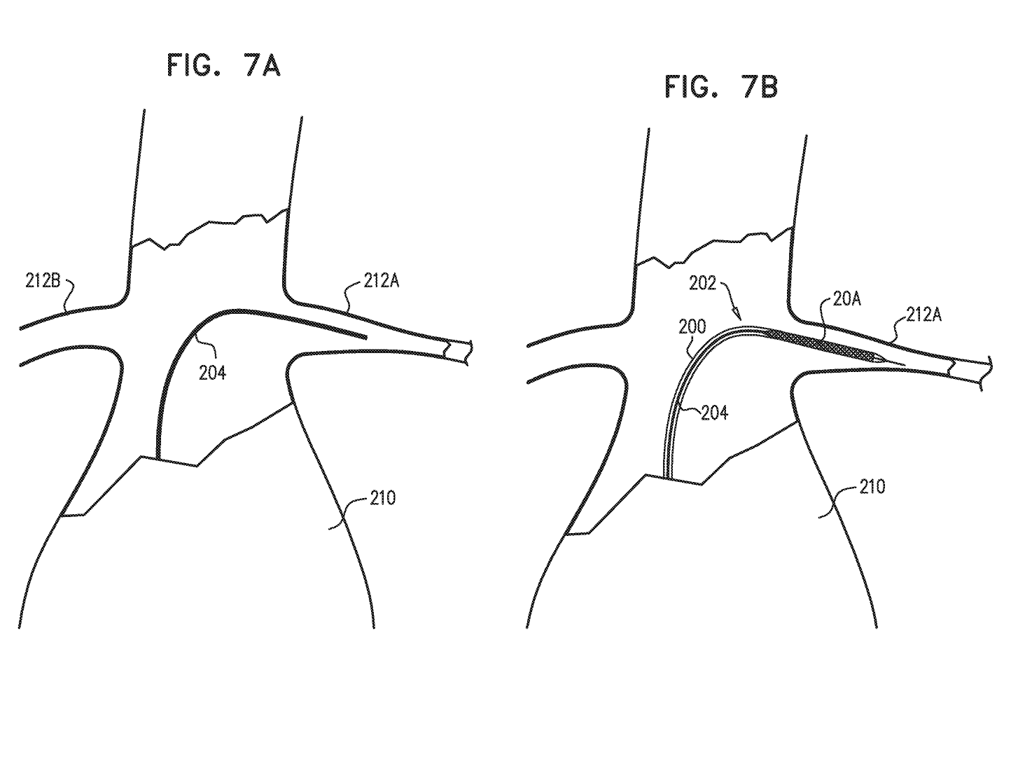

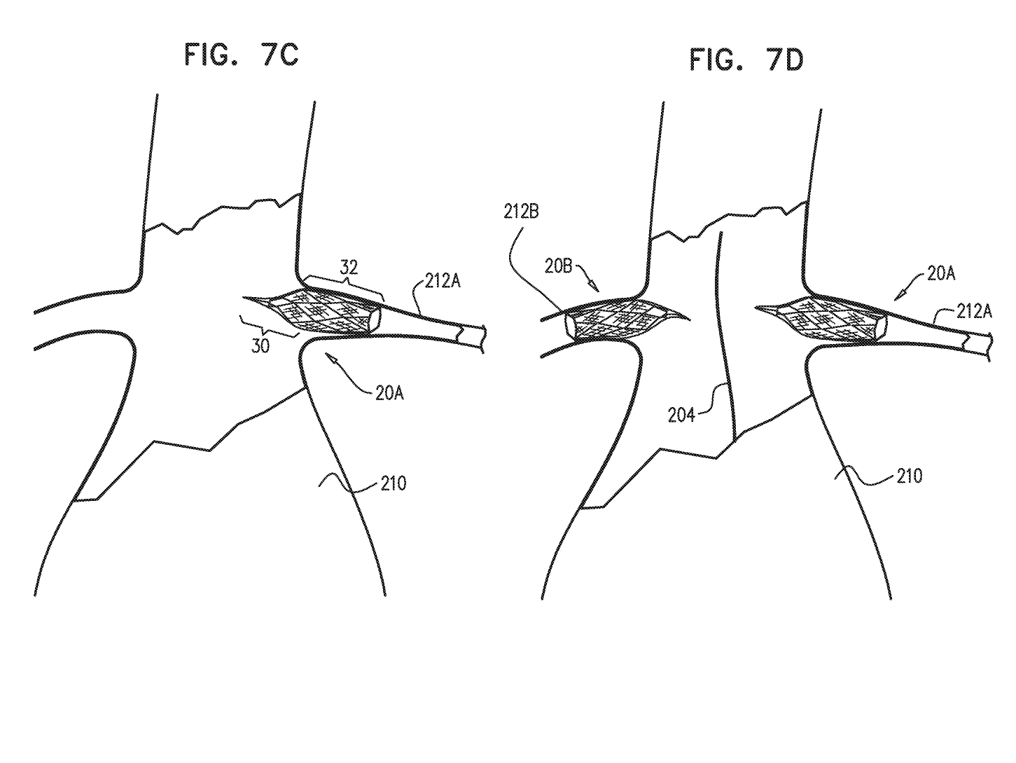

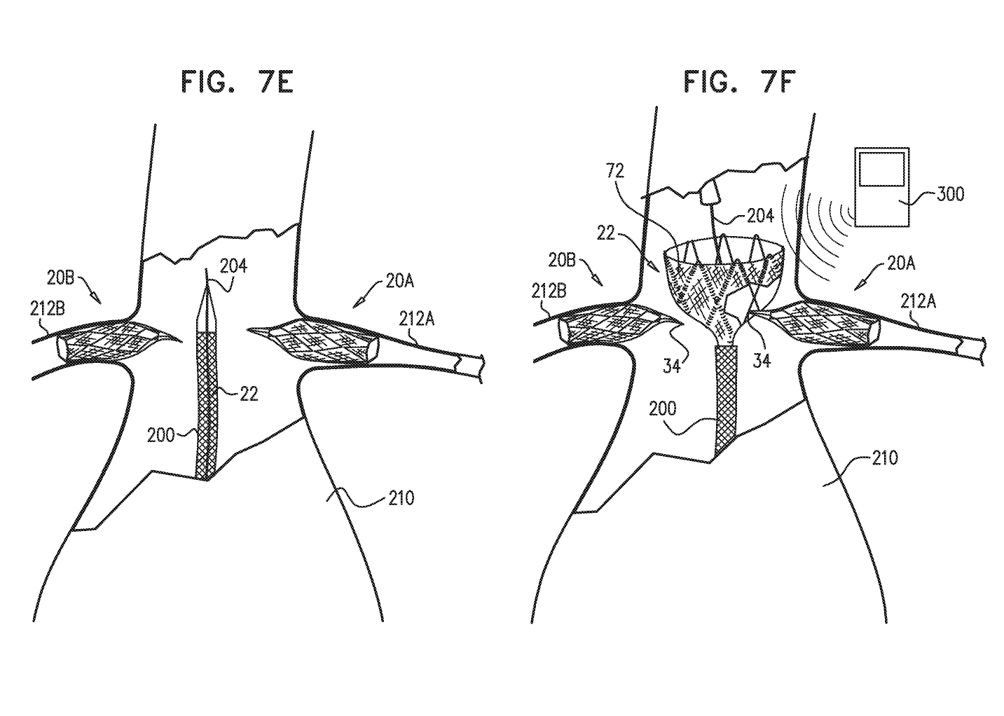

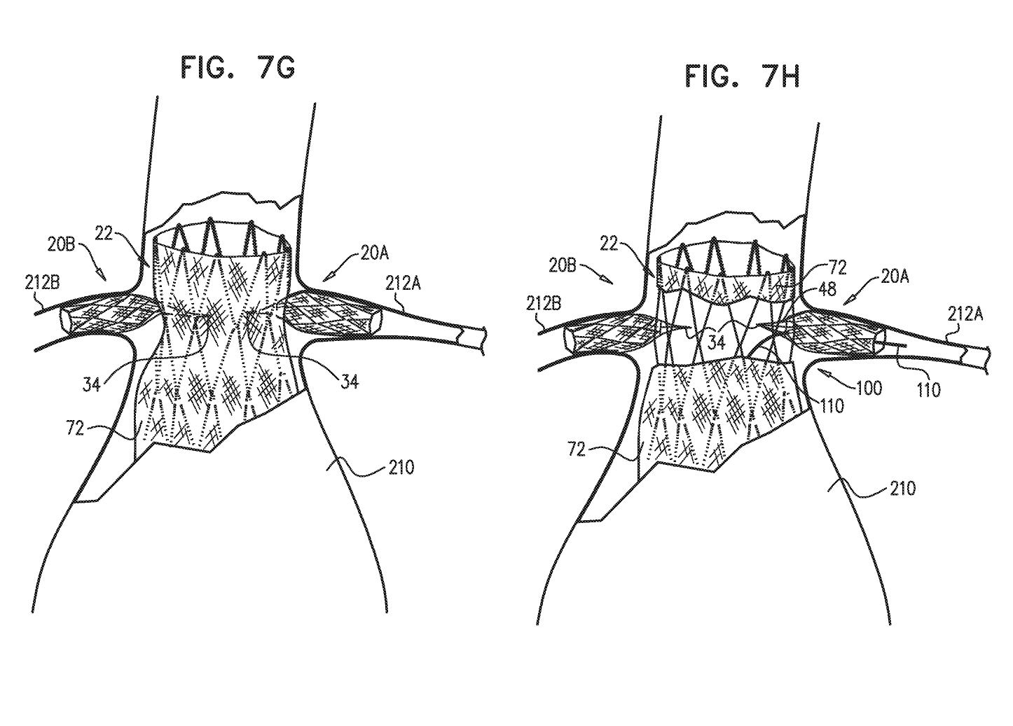

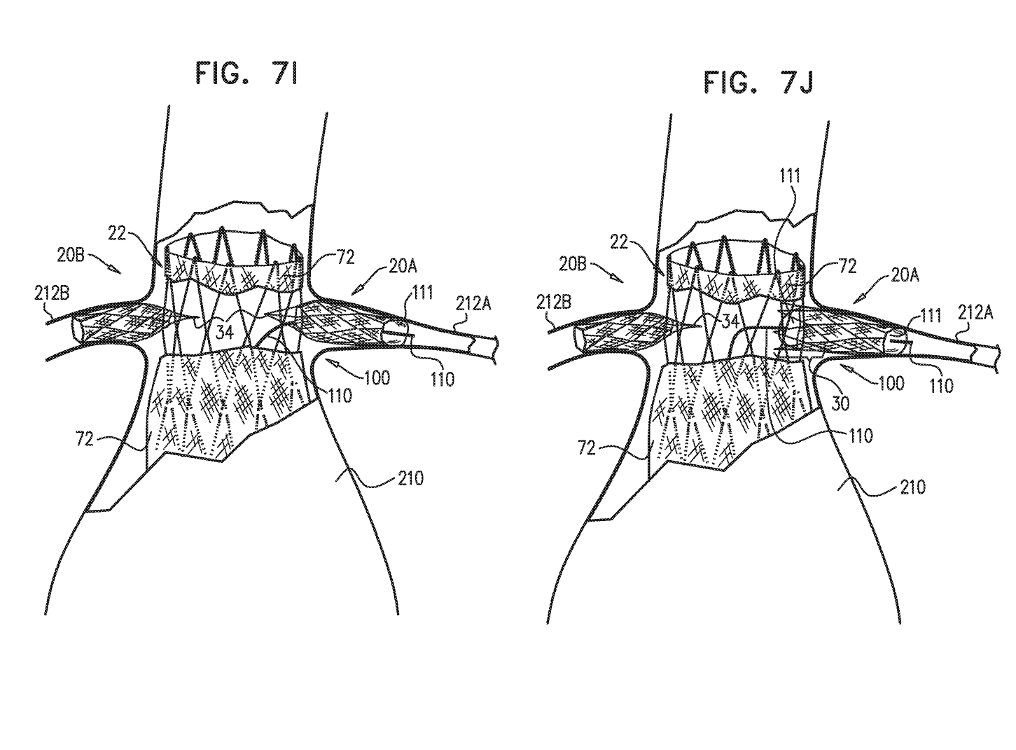

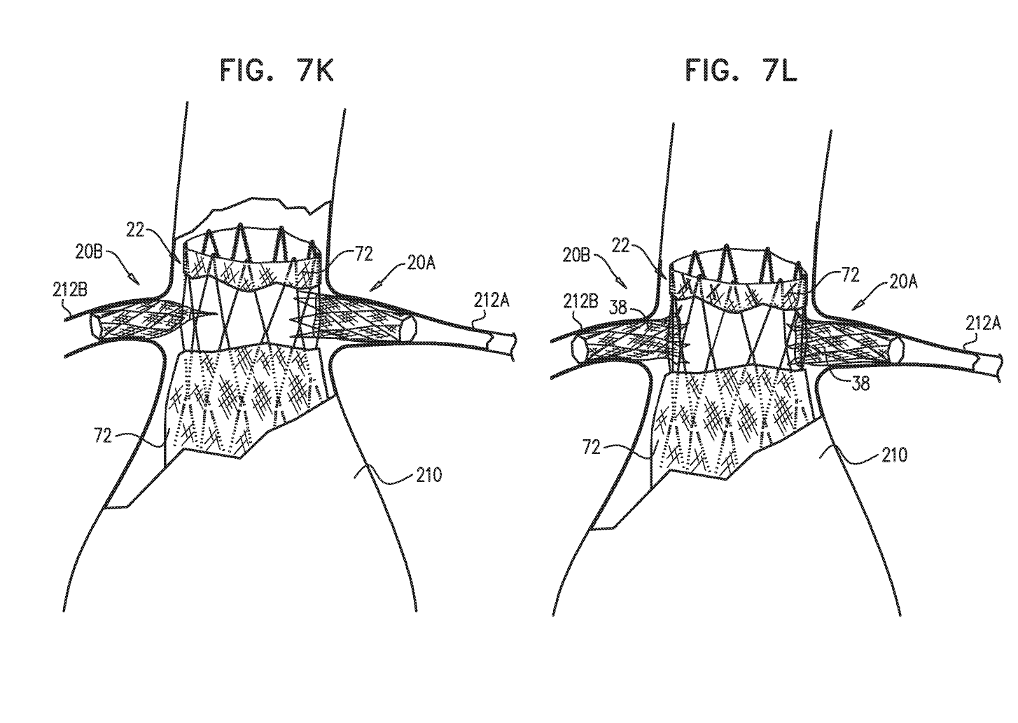

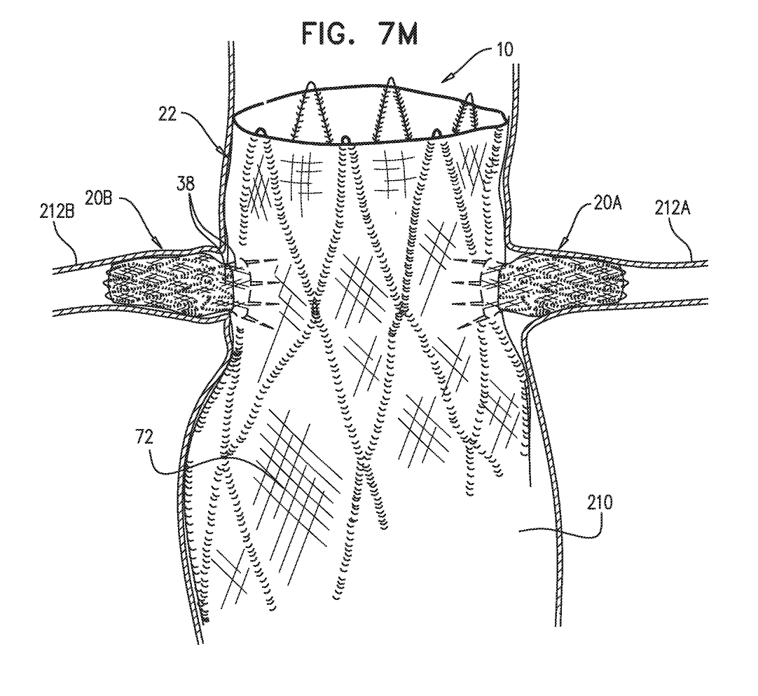

FIGS. 1 and 2 are schematic illustrations of multi-component stent-graft system 10 in disassembled and assembled states, respectively, in accordance with an application of the present invention. Multi-component stent-graft system 10 typically comprises a first branching endovascular stent-graft 20A and a main endovascular stent-graft 22, and, optionally, a second branching endovascular stent-graft 20B. Typically, for applications in which the second branching stent-graft is provided, the first and second branching stent-grafts are generally similar in configuration, although their dimensions may differ, depending on the target blood vessels. The stent-grafts are configured to assume radially-compressed states, such as when initially positioned in one or more delivery shafts of one or more delivery tools, as described hereinbelow with reference to FIGS. 7B and 7E, and to assume radially-expanded states upon being deployed from the delivery shaft(s), as described hereinbelow with reference to FIGS. 7C and 7F-G. In addition, a portion of each branching stent-graft is typically configured to assume a partially-radially-expanded state and a more-radially-expanded state, as described hereinbelow with reference to FIGS. 3A-C.

For some applications, the stent-grafts are relaxed in their radially-expanded states. For some applications, the stent-grafts are configured to be self-expanding. For example, they may be heat-set to assume the radially-expanded states. For some applications, stent-graft system 10 comprises more than two branching stent-grafts, such as exactly three, or exactly four, branching stent-grafts (configurations not shown).

FIG. 1 shows stent-graft system 10 in a disassembled state. Main stent-graft 22 is shown in its radially-expanded state. Each of branching stent-grafts 20A and 20B includes a proximal coupling portion 30 and, optionally, a distal body portion 32, as described in more detail hereinbelow with reference to FIGS. 3A-C. Distal body portion 32 is shown in its radially-expanded state. Coupling portion 30 is shown in a partially-radially-expanded state, in which the coupling portion defines a sharp tip 34 at a proximal end thereof. Sharp tip 34 is capable of puncturing a blood-impervious medical flexible sheet, such as a covering element 72 of main stent-graft 20.

FIG. 2 shows stent-graft system 10 in an assembled state. Coupling portions 30 of branching stent-grafts 20A and 20B are coupled to main stent-graft 22, so as to form blood-impervious seals between covering elements of the branching stent-grafts and covering element 72 of the main stent-graft. Coupling portions 30 are shown in more-radially-expanded states than the partially-radially-expanded states shown in FIG. 1.

The Branching Stent-grafts

FIGS. 3A-C are schematic illustrations of one of branching stent-grafts 20 in different levels of radial compression, in accordance with an application of the present invention. FIG. 3A shows branching stent-graft 20 initially placed in a tubular delivery shaft 40 (such as a catheter) in a radially-compressed state. FIG. 3B shows branching stent-graft 20 in a state assumed upon deployment from delivery shaft 40.

Branching stent-graft 20 comprises a support structure 36 and a covering element 38, which is securely attached to the support structure so as to at least partially cover (e.g., only partially cover) the support structure. Support structure 36 typically comprises a plurality of structural stent elements. For some applications, at least some of e.g., all of, the structural stent elements are interconnected (as shown in the figures), while for other applications, at least a portion of, e.g., all, of the structural stent elements are not interconnected (configuration not shown). For some applications, support structure 36 comprises a metal, such as a super-elastic alloy and/or a shape memory allow, e.g., Nitinol.

Covering element 38 serves as a blood flow guide through at least a portion of branching stent-graft 20. Covering element 38 (and covering element 72 of the main stent-graft) typically comprises at least one biologically-compatible substantially blood-impervious flexible sheet, which is attached (such as by stitching) to at least a portion of the support structure, on either side of the surface defined by the support structure. The flexible sheet may comprise, for example, a polymeric material (e.g., a polyester, or polytetrafluoroethylene), a textile material (e.g., polyethylene terephthalate (PET)), natural tissue (e.g., saphenous vein or collagen), or a combination thereof.

For applications in which branching stent-graft 20 includes distal body portion 32, a proximal end of the distal body portion is typically joined to a distal end of coupling portion 30. Distal body portion 32 is typically configured to transition to its radially-expanded state upon deployment of branching stent-graft 20 from the delivery shaft, as shown in FIG. 3B. For some applications, a distal end of distal body portion 32 coincides with the distal end of support structure 36.

Upon deployment of branching stent-graft 20 from the delivery shaft, coupling portion 30 is configured to transition to a partially-radially-expanded state, as shown in FIG. 3B. In this partially-radially-expanded state, coupling portion 30 defines sharp tip 34 at the proximal end of the support structure. For some applications, sharp tip 34 is oriented in a direction that is parallel to a central longitudinal axis 42 of branching stent-graft 20. Alternatively or additionally, the sharp tip is disposed within 1 mm of central longitudinal axis 42. For some applications, when the coupling portion is in the partially-radially-expanded state, the proximal end of the support structure is disposed within a circle 41 perpendicular to longitudinal axis 42 of the stent-graft, which circle has a diameter D1 of no more than 1 mm.

For some applications, the structural stent elements of support structure 36 include a plurality of proximal structural stent elements 50, which have respective proximal ends and are disposed around longitudinal axis 42 of coupling portion 30. The proximal ends of proximal structural stent elements 50 together define sharp tip 34 when coupling portion 30 is in the partially-radially-expanded state. Typically, support structure 36 is configured such that the proximal ends of proximal structural stent elements 50 together do not define sharp tip 34 when the coupling portion is in the more-radially-expanded state, as shown in FIG. 3C.

For some applications, proximal structural stent elements 50 that together define sharp tip 34 are continuations of all or a portion of the structural stent elements that define a more distal portion of coupling portion 30, and/or a portion of distal body portion 32. Typically, proximal structural stent elements 50 are distributed (e.g., uniformly) around the circumference of coupling portion 30, in order to give proximal portion a generally conical shape when proximal structural stent elements 50 come together at sharp tip 34. For example, a perimeter of coupling portion 30 may monotonically decrease from a distal end 44 thereof to a proximal end 46 thereof when the coupling portion is unconstrained in the partially-radially-expanded state.

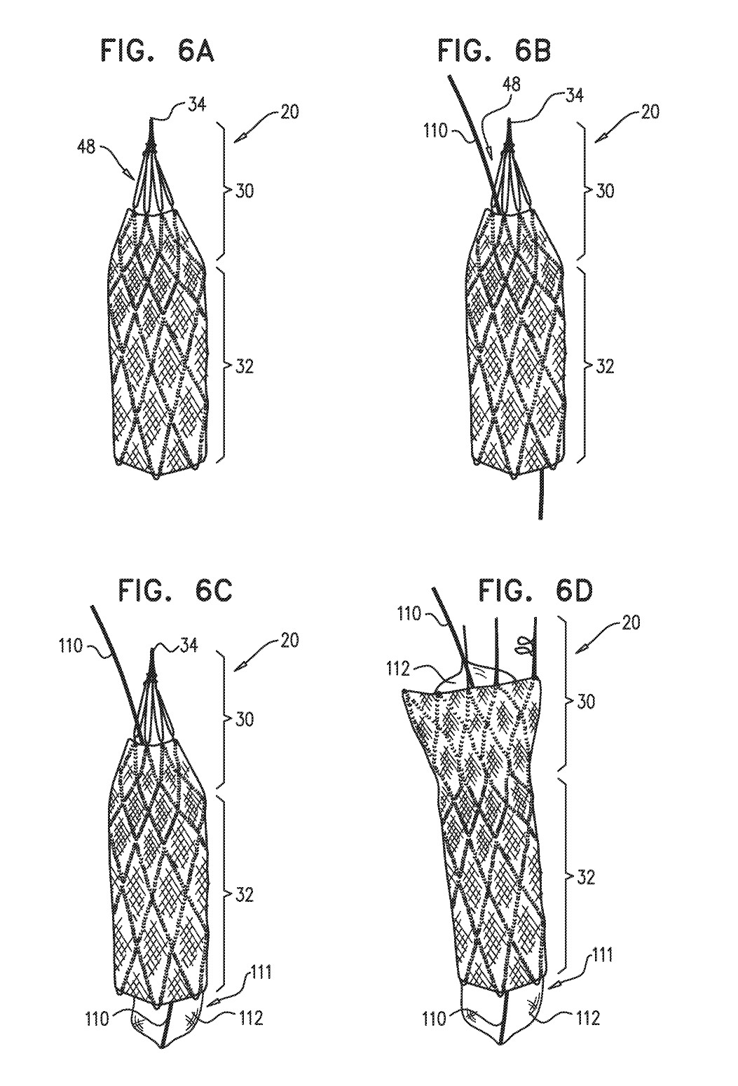

For some applications, covering element 38 covers a covered portion of coupling portion 30, which covered portion extends from distal end 44 of coupling portion 30 along between 20% and 100% (such as between 20% and 75%, or between 20% and 50%) of an axial distance D2 between distal end 44 and proximal end 46 of coupling portion 30, when the coupling portion is in the partially-radially-expanded state. Alternatively or additionally, for some applications, covering element 38 is shaped so define at least one non-covered portion 48 of a surface defined by coupling portion 30, which non-covered portion has a surface area of at least 1.5 mm2, such as at least 3 mm2, when the coupling portion is unconstrained in the partially-radially-expanded state, i.e., no forces are applied to the stent-graft by a delivery tool, walls of a blood vessel, or otherwise. The portion of the coupling portion that is not covered by covering element 38 may serve to allow access into branching stent-graft 20 for a coupling-end expansion tool 100, such as described hereinbelow with reference to FIGS. 6B and 7H-I.

Typically, coupling portion 30 is configured to be initially restrained in the partially-radially-expanded state upon the deployment of stent-graft 20 from delivery shaft 40. For some applications, a coupling-end restraining element 60 is provided, which is configured to initially restrain the proximal ends of proximal structural stent elements 50 that together define sharp tip 34. For some applications, coupling-end restraining element 60 is initially removably coupled to a portion of coupling portion 30, and coupling-end expansion tool 100 is configured to decouple the coupling-end restraining element from the portion of the coupling portion.

For some applications, stent-graft 20 further comprises coupling-end restraining element 60, which may be fixed to a portion of coupling portion 30. For some applications, coupling-end restraining element 60 comprises at least one curved element selected from the group consisting of: a ring, a helix, a coil, a spiral, and a corkscrew. The curved element is fixed to at least one (typically, exactly one) of proximal structural stent elements 50, typically within 2 mm of the proximal end of the at least one of the proximal structural stent elements. The other ones of proximal structural stent elements 50 are initially threaded through the curved element, such that coupling-end restraining element 60 initially restrains coupling portion 30 in the partially-radially-expanded state after the deployment of stent-graft 20 from delivery shaft 40. Alternatively or additionally, for some applications, coupling-end restraining element 60 comprises a biologically-compatible glue.

Alternatively, for some applications, proximal structural stent elements 50 are initially bundled together, so as to initially restrain coupling portion 30 in the partially-radially-expanded state after the deployment of the stent-graft from the delivery shaft. For these applications, coupling-end restraining element 60 is typically not provided.

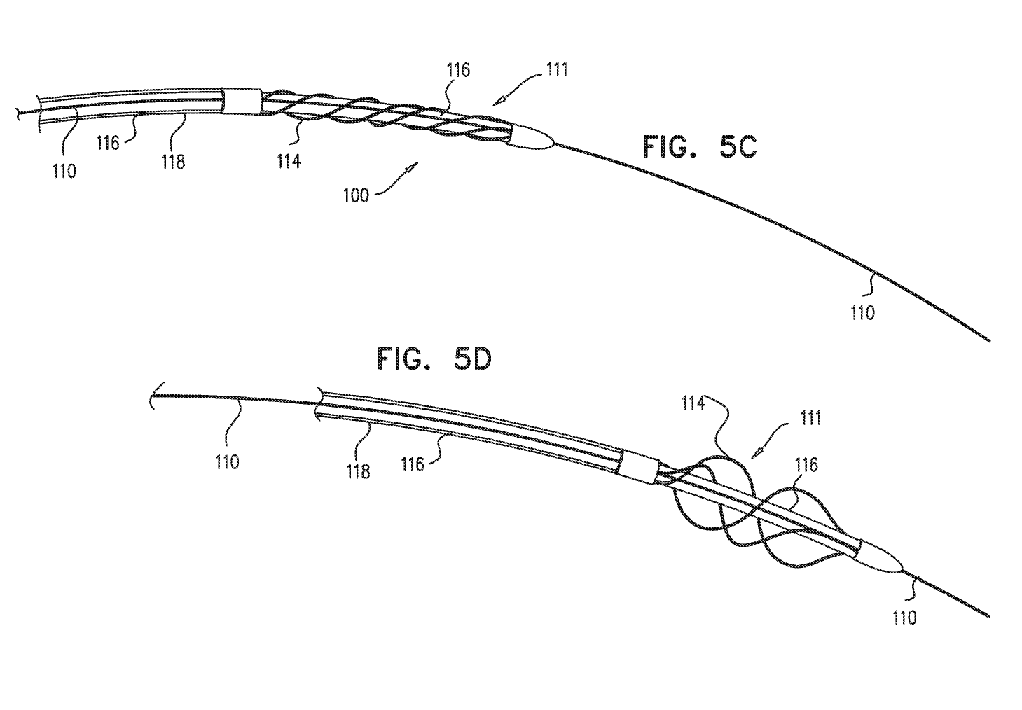

As shown in FIG. 3C, coupling portion 30 is configured to be transitioned from the partially-radially-expanded state to a more-radially-expanded state, such as using coupling-end expansion tool 100, described hereinbelow with reference to FIGS. 5A-D. In this more-radially-expanded state, coupling-end restraining element 60 no longer restrains coupling portion 30 to define sharp tip 34. For some applications, coupling portion 30 is outwardly flared in a proximal direction when the coupling portion is unconstrained in the more-radially-expanded state, as shown in FIG. 3C. Alternatively, the coupling portion is not flared (configuration not shown).

For some applications, the more-radially-expanded state is the resting state of coupling portion 30, i.e., the state that the coupling portion is configured (e.g., heat-set) to assume when not restrained in the partially-radially-expanded state, such as by coupling-end restraining element 60.

For some applications, coupling portion 30 has a greatest perimeter of at least 10 mm, no more than 50 mm, and/or between 10 and 50 mm, when the coupling portion is unconstrained in the more-radially-expanded state. For some applications, coupling portion 30 has an axial length of at least 0.5 cm, no more than 4 cm, and/or between 0.5 and 4 cm, when the coupling portion is in the more-radially-expanded state. For some applications, coupling portion 30 has an axial length of at least 0.3 cm, no more than 4 cm, and/or between 0.3 and 4 cm, when the coupling portion is in the partially-radially-expanded state.

For some applications, coupling portion 30 has (a) a partially-radially-expanded volume of at least 30 mm3, such as at least 200 mm3, when in the partially-radially-expanded state, and (b) a more-radially-expanded volume equal to at least 300% of the partially-radially-expanded volume, when unconstrained in the more-radially-expanded state.

For applications in which stent-graft 20 includes distal body portion 32, the distal body portion is generally tubular when unconstrained in the radially-expanded state. For some applications, distal body portion 32 has a greatest perimeter of at least 10 mm, no more than 50 mm, and/or between 10 and 50 mm, when the distal body portion is unconstrained in the radially-expanded state. For some applications, distal body portion 32 has an axial length of at least 0.5 cm, no more than 5 cm, and/or between 0.5 and 5 cm, when the distal body portion is in the radially-expanded state. For some applications, stent-graft 20 has an axial length of at least 0.5 cm, no more than 6 cm, and/or between 0.5 and 6 cm, when the distal body portion is in the radially-expanded state and the coupling portion is in the more-radially-expanded state. For some applications, covering element 38 covers at least 75% of an axial length of distal body portion 32, when the distal body portion is in the radially-expanded state.

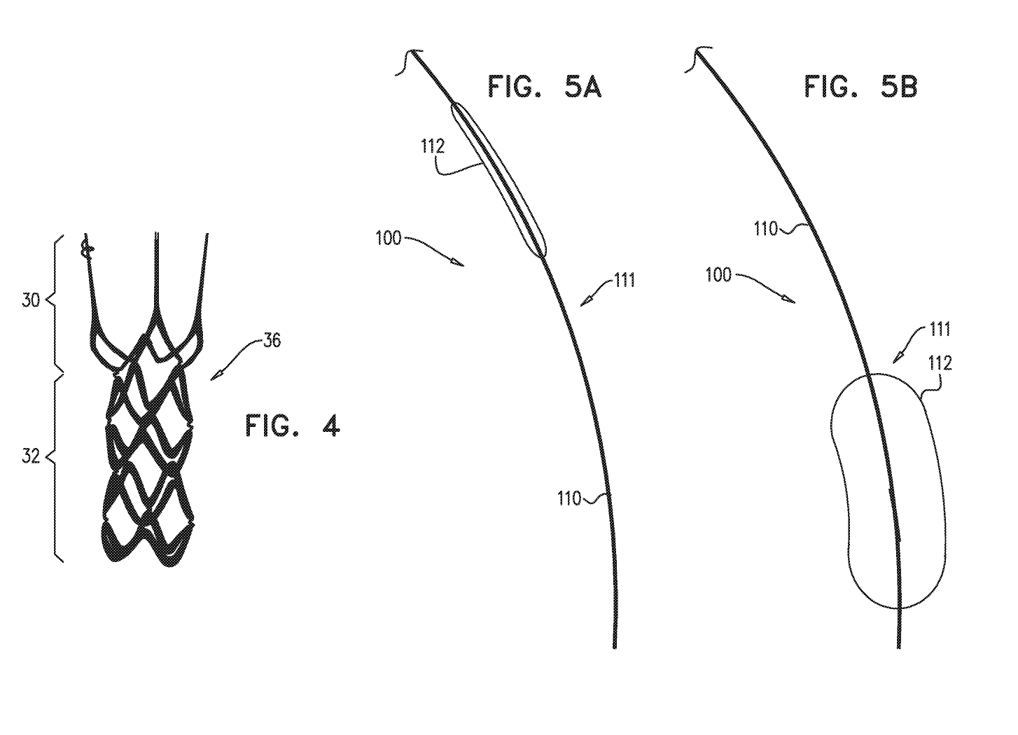

Reference is made to FIG. 4, which is a schematic illustration of support structure 36 unconstrained in the more-radially-expanded state, in accordance with an application of the present invention. For some applications, coupling portion 30 is outwardly flared in a proximal direction when the coupling portion is unconstrained in the more-radially-expanded state, as shown in FIG. 4. Alternatively, the coupling portion is not flared (configuration not shown).

The Main Stent-Graft

Reference is again made to FIG. 1. Main stent-graft 22 comprises a main generally tubular support structure 70 and main covering element 72, which is securely attached to the support structure so as to at least partially cover (e.g., only partially cover) the support structure. Support structure 70 typically comprises a plurality of structural stent elements. For some applications, at least some of, e.g., all of, the structural stent elements are interconnected (as shown in the figures), while for other applications, at least a portion of e.g., all, of the structural stent elements are not interconnected (configuration not shown). Main covering element 72 serves as a blood flow guide through at least a portion of the main stent-graft.

As mentioned above, main stent-graft 22 is configured to assume radially-compressed and radially-expanded states. For some applications, when unconstrained in the radially-expanded state, the main stent-graft has a greatest perimeter that is at least 150%, such as at least 200% or at least 300%, of a greatest perimeter of branching stent-graft 20 when coupling portion 30 is unconstrained in the more-radially-expanded state. For some applications, the greatest perimeter of the main stent-graft 22 is at least 6 cm, no more than 16 cm, and/or between 6 and 16 cm.

For some applications, main stent-graft 22 implements one or more of the techniques described in the patent applications incorporated by reference hereinbelow. For example, the main stent-graft may utilize one or more of the configurations of aortic stent-grafts described in these patent applications.

### The Coupling-End Expansion Tool

Reference is made to FIGS. 5A-D, which are schematic illustrations of coupling-end expansion tool 100, in accordance with respective applications of the present invention. Coupling-end expansion tool 100 comprises a radially-expandable member 111, and, typically, a guidewire 110, over which the radially-expandable member is advanced. Typically, tool 100 additionally comprises one or more tubes for facilitating such advancement, as is known in the art. For clarity of illustrations, these tubes are not shown in the figures.

In the configuration of coupling-end expansion tool 100 shown in FIGS. 5A-B, radially-expandable member 111 comprises an inflatable element 112, such as a balloon, which is typically filled with a fluid, such as saline solution, in order to expand the element. An inflation tube is provided for providing the fluid (not shown).

In the configuration of coupling-end expansion tool 100 shown in FIGS. 5C-D, radially-expandable member 111 comprises a radially-expandable metal wireframe 114, which may, for example, comprise a shape memory alloy, e.g., Nitinol. For example, the wireframe may comprise one or more elongated helical wires, which are configured such that when ends of radially-expandable member 111 are brought closer to each other (such that the member becomes axially-compressed), the member radially expands, and when the ends are brought farther from each other (such that the member becomes axially-extended), the member radially contracts. For some applications, in order to advance wireframe 114 along guidewire 110 and to control the axial distance between the ends of the wireframe, coupling-end expansion tool 100 further comprises inner and outer tubes 116 and 118. Inner tube 116 passes through the wireframe, and is coupled to the distal end of the wireframe. Outer tube 118 surrounds inner tube 116, and is coupled to the proximal end of the wireframe. Distally pushing both tubes 116 and 118 together advances wireframe 114 distally along guidewire 110. After the wireframe reaches the desired location near the distal end of the guidewire, distally pushing outer tube 118, while holding inner tube 116 stationary, brings the ends of the wireframe toward each other, thereby radially expanding the wireframe. The use of metal wireframe 114, rather than inflatable element 12, may eliminate any risk that structural elements of branching stent-graft 20 may puncture inflatable element 112.

FIGS. 5A and 5C show radially-expandable member 111 sliding over guidewire 110, while in a non-radially-expanded state. FIGS. 5B and SD show the radially-expandable member after it has arrived near the distal end of guidewire 110 and transitioned to a radially-expanded state.

Reference is made to FIGS. 6A-D, which are schematic illustrations of a procedure for expanding coupling portion 30 of branching stent-graft 20, in accordance with an application of the present invention. Although radially-expandable member 111 is shown comprising inflatable element 112, described hereinabove with reference to FIGS. 5A-B, the radially-expandable member may alternatively or additionally comprise radially-expandable metal wireframe 114, described hereinabove with reference to FIGS. 5C-D, and/or another radially-expandable member.

FIG. 6A shows branching stent-graft 20 in its partially-radially-expanded state, and with coupling portion 30 configured to define non-covered portion 48.

FIG. 6B shows the introduction of guidewire 110 of coupling-end expansion tool 100 through non-covered portion 48 of coupling portion 30. The guidewire is advanced at least partially into the interior of branching stent-graft 20, optionally into distal body portion 32, for applications in which the distal body portion is provided, and further optionally out of a distal end of the stent-graft.

Radially-expandable member 111, while in its non-radially-expanded state, is advanced over guidewire 110 (such as using a tube, as known in the art), until the balloon is at least partially in coupling portion 30, and, optionally, partially in distal portion 32.

FIG. 6C shows the radially-expandable member partially transitioned to its radially-expanded state (inflatable element 112 is partially inflated). FIG. 6D shows the radially-expandable member radially expanded sufficiently to transition coupling portion 30 from its partially-radially-expanded state to its more-radially-expanded state. Typically, in the more-radially-expanded state, coupling portion 30 no longer defines sharp tip 34, as shown in FIG. 6D. Subsequently, radially-expandable member 111 is radially contracted (e.g., deflated, if inflatable element 112, or axially-extended, if wireframe 114), and withdrawn from branching stent-graft 20.

Alternatively, instead of using this over-the-wire technique, inflatable element 112 is advanced using a rapid-exchange technique, as is known in the art.

For some applications, coupling-end restraining element 60 is initially removably coupled to a portion of coupling portion 30, and coupling-end expansion tool 100 is configured to decouple coupling-end restraining element 60 from the portion of coupling portion 30.

For some applications, coupling-end restraining element 60 comprises one or more of proximal structural stent elements 50 that define sharp tip 34, described hereinabove with reference to FIGS. 3B and 3C, and coupling-end expansion tool 100 is configured to transition coupling portion 30 to its more-radially-expanded state by breaking proximal structural stent elements 50 of coupling-end restraining element 60.

### An Exemplary Deployment Procedure

Reference is made to FIGS. 7A-M, which are schematic illustrations of an exemplary transluminal delivery procedure for implanting multi-component stent-graft system 10, in accordance with an application of the present invention.

In this exemplary procedure, the stem-grafts of system 10 are transvascularly (typically percutaneously) introduced into the aorta via one of the iliac arteries, while the stent-grafts are positioned in one or more delivery shafts 200 of a delivery tool 202 in their radially-compressed states. As shown in FIG. 7A, the exemplary procedure begins with the advancing of a guidewire 204 up an aorta 210 and into a first one of the renal arteries, such as a left renal artery 212A.

First branching stent-graft 20A is initially positioned in its radially-compressed state within delivery shaft 200 of delivery tool 202, typically near a distal end of the delivery shaft (e.g., such that at least one end of stent-graft 20A is within a distance of the distal end, which distance equals the sum of 2 cm and an axial length of the first branching stent-graft). Delivery shaft 200 is advanced over guidewire 204, until first branching stent-graft 20A is partially disposed in left renal artery 212A and partially disposed in aorta 210 near the left renal artery, as shown in FIG. 7B. The guidewire is withdrawn, leaving delivery shaft 200 in place.

As shown in FIG. 7C, the first branching stent-graft is held in place as delivery shaft 200 is withdrawn, thereby delivering the first branching stent-graft from the delivery shaft. Optionally, techniques for holding the first stent-graft in place may be used that are described in a PCT application filed Nov. 30, 2010, entitled, “Multi-component stent-graft system for implantation in a blood vessel with multiple branches,” which is incorporated herein by reference, such as with reference to FIGS. 10 and 11A-E or FIGS. 12A-C thereof. Alternatively, the first branching stent-graft (and/or the second branching stent graft or the main stent-graft, as described hereinbelow) is delivered using an over-the-wire (OTW) approach, in which the guidewire is left in place until the stent-graft is expanded, and thereafter the guidewire is withdrawn.

First branching stent-graft 20A typically self-expands, until distal body portion 32 assumes its radially-expanded state, upon reaching its maximum unconstrained size, and/or being constrained from further expansion by the wall of the blood vessels. Coupling portion 30 expands to its partially-radially-expanded state, in which the coupling portion defines sharp tip 34 at the proximal end of support structure 36. A distal end of first branching stent-graft 20A is positioned in left renal artery 212A, and a proximal end of the first branching stent-graft, including sharp tip 34, extends into aorta 210.

As shown in FIG. 7D, second branching stent-graft 20B is delivered to the second renal artery, such as a right renal artery 212B, using the procedure described hereinabove with reference to FIGS. 7A-C. The same guidewire and/or delivery shaft may be used to deploy the second branching stent-graft as was used to deploy the first branching stent-graft, or a separate guidewire and/or delivery shaft may be used.

Also as shown in FIG. 7D, a guidewire (either the same guidewire 204 used to deploy the first and/or second branching stent-grafts, or a second guidewire) is advanced up aorta 210, at least slightly past the bifurcations of the renal arteries.

As shown in FIG. 7E, main stent-graft 22 is positioned in its radially-compressed state within an delivery shaft of a delivery tool (either the same delivery shaft 200 used to deploy the first and/or second branching stent-grafts, or a separate delivery shaft), typically near the distal end of the delivery shaft (e.g., such that at least one end of main stent-graft 22 is within a distance of the distal end, which distance equals the sum of 2 cm and an axial length of the main stent-graft). Delivery shaft 200 is advanced over guidewire 204, until main stent-graft 22 is partially disposed in aorta 210 such that respective axial portions of the main stent-graft is aligned with left and right renal arteries 212A and 212B.

As shown in FIGS. 7F and 7G, the main stent-graft is held in place as delivery shaft 200 is withdrawn, thereby delivering the main stent-graft from the delivery shaft. FIG. 7F shows the delivery shaft partially withdrawn, and the main stent-graft partially deployed, and FIG. 7G shows the delivery shaft fully withdrawn, and the main stent-graft fully deployed. Main stent-graft 22 typically self-expands, until it assumes its radially-expanded state, upon reaching its maximum unconstrained size, and/or being constrained from further expansion by the wall of the blood vessels.

As main stent-graft 22 transitions to its radially-expanded state, sharp tips 34 of first and second branching stent-grafts 20A and 20B puncture main covering element 72, thereby forming a fenestration in the main covering element, as shown in FIGS. 7F and 7G (FIG. 7F, as well as FIGS. 7H-L, include a cutaway portions for clarity of illustration). The outward radial force applied by support structures 36 of branching stent-grafts 20A and 2013 prevents further distal advancement of the branching stent-grafts in the renal arteries, thereby holding the sharp tips stationary such that they puncture the main covering element as the main covering element expands.

In order to transition coupling portion 30 of first branching stent-graft 20 from its partially-radially-expanded state to its more-radially-expanded state, coupling-end expansion tool 100 is advanced to the first branching stent-graft, typically through main stent-graft 22. As shown in FIG. 7H, guidewire 110 of expansion tool 100 is typically advanced through non-covered portion 48 of coupling portion 30 of the branching stent-graft (such as described in more detail hereinabove with reference to FIG. 6B).

As shown in FIG. 7I, radially-expandable member 111 is advanced over the guidewire into the branching stent-graft (such as described in more detail hereinabove with reference to FIGS. 5A-D and 6C).

As shown in FIG. 7J, radially-expandable member 111 is transitioned to its radially-expanded state, thereby transitioning coupling portion 30 from its partially-radially-expanded state to its more-radially-expanded state. Typically, in the more-radially-expanded state, coupling portion 30 no longer defines sharp tip 34. As coupling portion 30 transitions from the partially-radially-expanded state to the more-radially-expanded state, coupling portion 30 typically enlarges the fenestration previously made in main covering element 72 of main stent-graft 22 by sharp tip 34, as described hereinabove with reference to FIGS. 7F and 7G. A blood-impervious seal is formed between covering element 38 of first branching stent-graft 20A and main covering element 72 of main stent-graft 22. (The blood-impervious seal can perhaps be best seen in FIGS. 2 and 7M.) Coupling-end expansion tool 100 is withdrawn, as shown in FIG. 7K.

The coupling portion expansion procedure described with reference to FIGS. 7H-K is repeated for second branching stent-graft 20B. For some applications, the same coupling-end expansion tool 100 is used for expanding both branching stent-grafts, while for other applications, a second coupling-end expansion tool 100 is provided for expanding second branching stent-graft 20B. Upon expansion of proximal portion 30, a blood-impervious seal is formed between covering element 38 of second branching stent-graft 2013 and main covering element 72 of main stent-graft 22, as shown in FIG. 7L (with a cutaway portion) and FIG. 7M (without the cutaway portion), thereby completing the procedure. (The blood-impervious seal can also be seen in FIG. 2.)

Reference is again made to FIG. 7F. For some applications, an extracorporeal unit 300 is provided, which is configured to transmit energy to coupling portion 30. Coupling portion 30 is configured to convert the energy to thermal energy to increase a capability of the sharp tip. For some applications, the energy is transmitted wirelessly (e.g., electromagnetically, or using magnetic induction), and may comprise, for example, RF energy. Alternatively, the energy may be transmitted using one of the delivery tools described hereinabove, or coupling-end expansion tool 100.

For some applications of the present invention, a kit is provided that comprises:
main stent-graft 22 and first branching stent-graft 20A, and, optionally, second branching stent-graft 20B; or
first and second branching stent-grafts 20A and 20B, and, optionally, main stent-graft 22.

For some applications, the kit further comprises one or more coupling-end expansion tools 100 and/or one or more delivery tools 202.

For some applications, at least one of stent-grafts 20A, 20B, and 22 comprise one or more anchoring elements that extend radially outwardly when the stent-graft assumes its radially-expanded state. The anchoring elements anchor the stent-graft to a vascular wall, helping prevent dislodgement.

For some applications, stent-graft system 10 is used to treat an aneurysm, such as an aortic aneurism, or an aneurism of another blood vessel. For example, the aneurism may be of the sub-renal aorta, as shown in FIGS. 7A-M. For some applications, a method is provided that comprises identifying that a patient suffers from an aneurysm, such as an aortic aneurism, and, responsively to the identifying, implanting (for example, including, transvascularly introducing) one or more of the stent-grafts described herein, such as main stent-graft 22, first branching stent-graft 20A, and/or second branching stent-graft 20B. Techniques for identifying that a patient suffers from an aneurism are well known, and thus not described herein.

Although stent-grafts 20A, 20B, and 22 have sometimes been described hereinabove as being deployed at the branches of one or more renal arteries from the aorta, the stent-grafts may be used for stent-grafting the celiac and/or mesenteric (superior and inferior) arteries and, for some applications, may also be deployed at other branching body lumens. For example:
the main body lumen may be the aorta, and the branching body lumen may include the inferior or superior mesenteric arteries, or the celiac artery;
the main body lumen may be the aorta and the branching body lumens may include both iliac arteries; or
the main body lumen may be the aortic arch and the branching body lumen may include the brachiocephalic artery, the left common carotid artery, and/or the subclavian artery.

The scope of the present invention includes embodiments described in the following applications, which are assigned to the assignee of the present application and are incorporated herein by reference. In an embodiment, techniques and apparatus described in one or more of the following applications are combined with techniques and apparatus described herein:
PCT Application PCT/IL2008/000287, filed Mar. 5, 2008, which published as PCT Publication WO 2008/107885 to Shalev et al,
U.S. application Ser. No. 12/529,936, which published as US Patent Application Publication 2010/0063575 to Shalev et al.
U.S. Provisional Application 60/892,885, filed Mar. 5, 2007
U.S. Provisional Application 60/991,726, filed Dec. 2, 2007
U.S. Provisional Application 61/219,758, filed Jun. 23, 2009
U.S. Provisional Application 61/221,074, filed Jun. 28, 2009
PCT Application PCT/IB2010/052861, filed Jun. 23, 2010, published as PCT Publication WO 2010/150208
PCT Application PCT/IB2010/000564, filed Jul. 14, 2010, which published as PCT Publication WO 2011/007354
PCT Application PCT/IL2010/000917, filed Nov. 4, 2010, which published as PCT Publication WO 2011/055364
PCT Application PCT/IL2010/000999, filed Nov. 30, 2010, entitled, “Multi-component stent-graft system for implantation in a blood vessel with multiple branches,” which published as PCT Publication WO 2011/064782

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1-51. (canceled)

52. A method for treating a patient, the method comprising:
transvascularly introducing a tubular delivery shaft partially into a branching blood vessel that branches from a main blood vessel, while a branching stent-graft is positioned in the tubular delivery shaft in a radially-compressed state, which branching stent-graft includes a branching support structure and a branching covering element that at least partially covers a coupling portion of the branching support structure, the branching support structure including a plurality of structural stent elements, which include a plurality of proximal structural stent elements, which have respective proximal ends and are disposed around a longitudinal axis of the coupling portion;
deploying the branching stent-graft from the delivery shaft such that (a) a distal end of the branching stent-graft is positioned in the branching blood vessel, and a proximal end of the branching stent-graft extends into the main blood vessel, and (b) the coupling portion transitions to a partially-radially-expanded state, in which the proximal ends of the proximal structural stent elements together define a sharp tip at a proximal end of the branching support structure;
transvascularly introducing a main endovascular stent-graft into the main blood vessel in a radially-compressed state, which main stent-graft includes a main support structure and a main covering element attached to the main support structure so as to at least partially cover the main support structure;
after deploying the branching stent-graft, and while the sharp tip remains stationary, transitioning the main stent-graft to a radially-expanded state, such that the sharp tip punctures the main covering element as the main stent-graft transitions to the radially-expanded state, thereby forming a fenestration in the main covering element; and
after transitioning the main stent-graft, transitioning the coupling portion of the branching stent-graft from the partially-radially-expanded state to a more-radially-expanded state such that the proximal ends of the proximal structural stent elements together do not define the sharp tip, so as to form a blood-impervious seal between the branching covering element and the main covering element.

53. The method according to claim 52, wherein the main and branching blood vessels are an aorta and a renal artery, respectively, wherein transvascularly introducing the tubular delivery shaft comprises transvascularly introducing the tubular delivery shaft partially into the renal artery, and wherein transvascularly introducing the main endovascular stent-graft comprises transvascularly introducing the main endovascular stent-graft into the aorta in the radially-compressed state.

54. The method according to claim 52, further comprising identifying that the patient suffers from an aneurysm, wherein transvascularly introducing the main stent-graft comprises transvascularly introducing the main stent-graft responsively to the identifying.

55. The method according to claim 52, further comprising identifying that the patient suffers from an aneurysm, wherein transvascularly introducing the branching stent-graft comprises transvascularly introducing the branching stent-graft responsively to the identifying.

56. The method according to claim 52, wherein transitioning the coupling portion comprises enlarging the fenestration by transitioning the coupling portion.

57. The method according to claim 52, wherein, when the coupling portion is in the partially-radially-expanded state, the proximal end of the branching support structure is disposed within a circle perpendicular to a longitudinal axis of the branching stent-graft, which circle has a diameter of no more than 1 mm.

58. The method according to claim 52, wherein the covering element is shaped so define at least one non-covered portion of a surface of the coupling portion, which non-covered portion has a surface area of at least 1.5 mm2, when the coupling portion is unconstrained in the partially-radially-expanded state.

59-60. (canceled)

61. The method according to claim 52, wherein the coupling portion is configured to be initially restrained in the partially-radially-expanded state upon the deploying of the branching stent-graft from the delivery shaft.

62. The method according to claim 61, wherein the branching stent-graft further includes a coupling-end restraining element, and wherein deploying the branching stent-graft comprises deploying the branching stent-graft such that the coupling-end restraining element initially restrains the proximal ends of the proximal structural stent elements that together define the sharp tip.

63. The method according to claim 62,
wherein the coupling-end restraining element includes at least one curved element selected from the group consisting of: a ring, a helix, a coil, a spiral, and a corkscrew, which curved element is fixed to at least one of the proximal structural stent elements within 2 mm of the proximal end of the at least one of the proximal structural stent elements, and
wherein the other ones of the proximal structural stent elements are initially threaded through the curved element, such that the coupling-end restraining element initially restrains the coupling portion in the partially-radially-expanded state after the deploying of the branching stent-graft from the delivery shaft.

64. The method according to claim 52, wherein the proximal structural stent elements are initially bundled together, so as to initially restrain the coupling portion in the partially-radially-expanded state after the deploying of the branching stent-graft from the delivery shaft.

65. The method according to claim 52, wherein transitioning the coupling portion comprises using a coupling-end expansion tool to transition the coupling portion from the partially-radially-expanded state to the more-radially-expanded state.

66. The method according to claim 65, wherein the covering element is shaped so define at least one non-covered portion of the coupling portion when the coupling portion is unconstrained in the partially-radially-expanded state, and wherein using the coupling-end expansion tool comprises passing the coupling-end expansion tool through the non-covered portion.

67. The method according to claim 52, wherein the branching support structure is shaped so as to further define a distal body portion, a proximal end of which is joined to a distal end of the coupling portion, and wherein deploying the branching stent comprises deploying the branching stent such that the distal body portion transitions to a radially-expanded state.

68. The method according to claim 52, wherein the branching support structure is self-expanding.

69. (canceled)

70. The method according to claim 52 wherein the coupling portion is configured to be initially restrained in the partially-radially-expanded state upon the deploying of the branching stent-graft from the delivery shaft.

71. The method according to claim 52
wherein the branching stent-graft is a first branching stent-graft, the coupling portion is a first coupling portion, the branching blood vessel is a first branching blood vessel, the branching support structure is a first branching support structure, the sharp tip is a first sharp tip, the fenestration is a first fenestration, the seal is a first seal, the plurality of structural stent elements is a first plurality of structural stent elements, the plurality of proximal structural stent elements is a first plurality of proximal structural stent elements, the respective proximal ends are first respective proximal ends, and longitudinal axis of the coupling portion is a first longitudinal axis of the first coupling portion,
wherein the method further comprises, before transitioning the main stent-graft to the radially-expanded state, transvascularly introducing and deploying a second branching stent-graft such that (a) a distal end of the second branching stent-graft is positioned in a second branching blood vessel that branches from the main blood vessel, and a proximal end of the second branching stent-graft extends into the main blood vessel, and (b) a second coupling portion of the second branching stent-graft transitions to a partially-radially-expanded state, in which second proximal ends of second proximal structural stent elements of a second branching support structure of the second branching stent-graft together define a second sharp tip at a proximal end of the second branching support structure of the second branching stent-graft,
wherein transitioning the main stent-graft comprises, after deploying the first and the second branching stent-grafts, and while the first and the second sharp tips remain stationary, transitioning the main stent-graft to the radially-expanded state, such that the first and second sharp tips puncture the main covering element as the main stent-graft transitions to the radially-expanded state, thereby forming the first fenestration and a second fenestration, respectively, in the main covering element, and
wherein the method further comprises, after transitioning the main stent-graft, transitioning the second coupling portion of the second branching stent-graft from the partially-radially-expanded state to a more-radially-expanded state such that the second proximal ends of the second proximal structural stent elements together do not define the second sharp tip, so as to form a second blood-impervious seal between the second branching covering element and the main covering element.
